(19) United States
(12) Patent Application Publication
(10) Pub. No.: US 2024/0425649 A1
(43) Pub. Date: Dec. 26, 2024

(54) SILICON POLYMER AND PREPARATION METHOD THEREFOR AND USE THEREOF

(71) Applicants: Guangzhou Panyu Polytechnic, Guangzhou (CN); Longway Technology (Guangzhou) Co., Ltd., Guangzhou (CN)

(72) Inventors: Mengyi XU, Guangzhou (CN); Rujing ZHENG, Guangzhou (CN); Peng ZHENG, Guangzhou (CN)

(21) Appl. No.: 18/734,609

(22) Filed: Jun. 5, 2024

(30) Foreign Application Priority Data
Jun. 15, 2023 (CN) 202310707442.9

(51) Int. Cl. C08G 77/26; A61K 8/898; A61Q 5/00; A61Q 5/02; C08G 77/08; C08G 77/46

(52) U.S. Cl. CPC C08G 77/26; A61K 8/898; A61Q 5/002; A61Q 5/02; C08G 77/08; C08G 77/46

(57) ABSTRACT

The present invention discloses a silicon polymer, a preparation method therefor and use thereof. The silicon polymer of the present invention has a structural formula shown as formula (I):

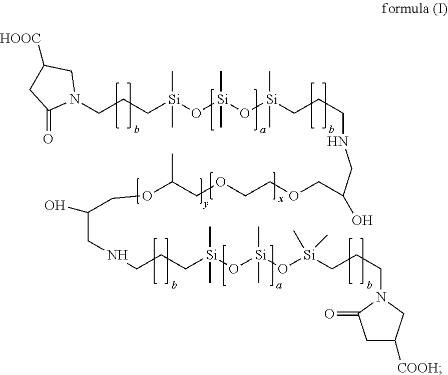

wherein 25≤a≤100, 0≤b≤8;
the formula (I) comprises a structure of

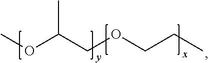

and the structure of

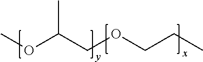

is derived from

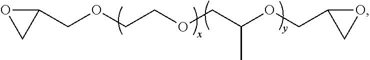

wherein the

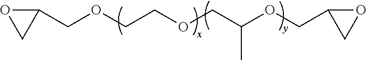

has a molecular weight of 1700-4400. The silicon polymer of the present invention can not only improve the affinity to hair, but also enhance the softness of hair, repair hair cuticle, make the surface of hair smoother, and weaken the hydrogen bonding force between polypeptide chains in hair.

### TECHNICAL FIELD

The present invention relates to the technical field of organic macromolecular compound, and specifically relates to a silicon polymer, and a preparation method therefor, and use thereof.

### BACKGROUND

Human hair has three morphological components mainly, i.e. the stratum corneum (the outermost thin shell of several concentric layers), cortex (the main body of hair), and medulla (the thin central core), among which the stratum corneum and cortex endow hair with mechanical properties, making it prone to waves, curls, or kinks. Generally speaking, straight hair can be similar to a rod with a circular cross-section, wavy hair has a cross-section which is compressed into an oval, curly hair can appear a slender elliptical cross-section which further compressed, and kinky hair has a flatter cross-section.

The primary component of hair is cross-linked protein keratin, wherein keratin intermediate filaments (KIFs) of the α-spiral type I and the α-spiral type II with a molecular weight of approximately 45-60 kDa is embedded into the amorphous matrix of keratin-associated proteins (KAPs) with a molecular weight ranging from 20-30 kDa (M. A. Rogers, L. Langbein, S. Praetzel-Wunder, H. Winter, J. Schweizer, J. Int Rev Cytol. 2006; 251:209-6). Both the intramolecular and intermolecular disulfide bonds provided by cystine contribute to maintaining the cytoskeletal protein network of the cytoskeleton. In addition to disulfide conjugates, ion bonding or salt bridges that pair various amino acids found in hair proteins also have a significant impact on the appearance of hair strands.

In daily life, people attach great importance to the care of hair after styling treatment (such as perm, dye, etc.). For example, existing technologies have disclosed a modified amino silicone oil, a preparation method, and use thereof in the preparation of protein-repair hair conditioners, wherein amino silane coupling agents, γ-aminopropyl methyl diethoxy silane and 3-aminopropyl triethoxy silane, are connected to polysiloxane to form a modified amino silicone oil having a network structure, which improves its encapsulation ability. At the same time, its nodes contain a large amount of amino groups, further enhancing its adhesion ability on the surface of hair and giving it long-lasting flexibility. Although the physical deposition of such modified silicone oil on the hair surface (stratum corneum) is beneficial for improving the surface properties (smoothness and friction) of hair, it cannot improve the softness of hair and cannot repair damaged hair cuticle.

### SUMMARY

An objective of the present invention is to provide a silicon polymer in order to overcome the defects and deficiencies that the existing modified silicone oil for hair treatment is difficult to effectively improve the softness of hair and to repair the damaged hair cuticle.

Another objective of the present invention is to provide a preparation method for the silicon polymer.

Another objective of the present invention is to provide a silicon polymer mixture.

Another objective of the present invention is to provide use of the silicon polymer or the silicon polymer mixture in preparing hair care products.

Another objective of the present invention is to provide a shampoo including the silicon polymer.

Another objective of the present invention is to provide a hair mask including the silicon polymer.

The above mentioned objectives of the present invention are achieved by the following technical solutions:

A silicon polymer, having a structural formula shown as formula (I):

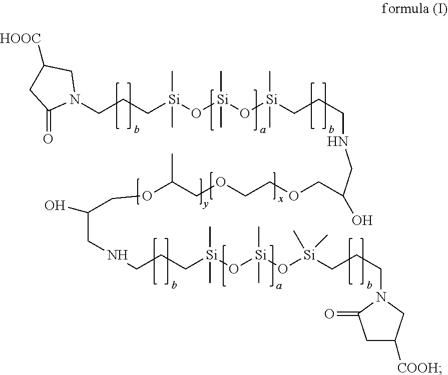

wherein 25≤a≤100, 0≤b≤8;
the formula (I) includes a structure of

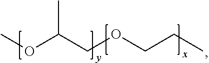

and the structure of

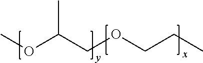

is derived from

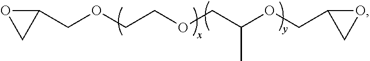

wherein the

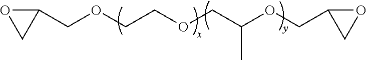

has a molecular weight of 1700-4400.

The silicon polymer of the present invention has both dimethylsilane segments and polyether segments, with pyrrolidone carboxylic acid for end capping. Particularly, ether bonds on the polyether segments are hydrophilic and polar which can improve the hydrophilic performance of silicon polymer, enhance the affinity to keratin, and further improve the affinity to hair. The dimethylsilane segments at both ends of the polyether segments are hydrophobic which provide repulsive force between the hairs and further enhance the fluffiness of hair. The pyrrolidone structures at both ends of the silicon polymer have good affinity to keratin, can be tightly attached to the damaged hair cuticle, and repair the hair cuticle, so that the hair surface is smoother, hydrogen boding between polypeptide chains among the hair is inhibited, and friction between the hairs is reduced so as to enhance the softness of hair.

The magnitude of a has an impact on residence property of the silicon polymer on hair. The greater the a, the better the residence property of the silicon polymer on hair, and the softer the hair. Improvement of the silicon polymer in softness and smoothness of the hair can be limited if a is too small; while tackiness of hair may be increased if a is too large.

The magnitude of b has a direct impact on a segment length of the pyrrolidone structure connected to the backbone —Si—O—Si— in the silicon polymer, and particularly, anchoring effect of the pyrrolidone structure on the damaged hair and affinity of the silicon polymer to keratin may be reduced when the segment length is too long, thereby worsening the repair effect.

By controlling a ration of x to y in the silicon polymer, relativeness of hydrophilicity and lipophilicity of the silicon polymer is controlled, thus meeting the practical application requirements of various hair care products. When x>y, the silicon polymer has a relatively stronger hydrophilicity which can be used in cosmetics that use water as a main solvent, such as shampoo. In such case, preferably, x is greater than y in the silicon polymer. When x<y, the silicon polymer has a relatively stronger lipophilicity which can be used in hair cosmetics in which oily components accounts more, such as hair mask and conditioner. In such case, preferably, x is less than y in the silicon polymer.

The present invention further provides a preparation method for the silicon polymer, including the following steps:
step S1, dissolving a double amino-terminated silicone oil in an organic solvent, then slowly adding a double epoxy-terminated polyether at −20° C. to 60° C. to perform a thermostatic reaction for 2-8 h for obtaining a reaction mixture A; and
step S2, adding a catalyst to the reaction mixture A obtained in step S1 and mixing uniformly, then removing the organic solvent, increasing a temperature of the reaction system to 100° C. to 150° C., subjecting the reaction system to a thermostatic reaction for 2-8 h for obtaining a reaction mixture B, and subjecting the reaction mixture B to separation and purification to obtain the silicon polymer;
wherein the double amino-terminated silicone oil is prepared by a dehydration condensation reaction of octamethylcyclotetrasiloxane and a reactant S, the reactant S is selected from a group consisting of Bis(aminoethyl) tetramethyl disiloxane, Bis(aminopropyl) tetramethyl disiloxane, Bis(aminobutyl) tetramethyl disiloxane, Bis(aminopentyl) tetramethyl disiloxane, Bis(aminohexyl) tetramethyl disiloxane, Bis(aminoheptyl) tetramethyl disiloxane, Bis(aminooctyl) tetramethyl disiloxane, Bis(aminononyl) tetramethyl disiloxane, and Bis(aminodecyl) tetramethyl disiloxane;
wherein the double epoxy-terminated polyether has a structural formula of

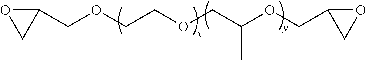

and a molecular weight of 1700-4400.

Particularly, the separation and purification of step S2 is as follows:
adding a strong base (5 wt %-20 wt % alcoholic solution) to the reaction mixture B after cooling the reaction mixture B of step S2, then stirring the reaction system at a heating condition of 50° C. to 80° C. and adding water thereto, adjusting a pH of the reaction system to 2-3.5, performing separation and purification to obtain the silicon polymer.

The slowly adding refers to controlling the adding within 1.5 to 5 hours, so as to reduce side reactions and enhance the consistency of molecular weight of product.

Particularly, the double amino-terminated silicone oil is prepared by the following method:
adding the octamethylcyclotetrasiloxane and the reactant S to a reaction vessel and mixing uniformly, heating to 90° C. to 95° C. in an inert atmosphere, adding tetramethylammonium hydroxide as a catalyst to the reaction vessel to perform a thermostatic reaction for 6-10 h, then performing separation and purification to obtain the double amino-terminated polysiloxane.

Particularly, a molar ratio of the octamethylcyclotetrasiloxane to the reactant S is (12.5-50):1.

A silicon polymer mixture including the silicon polymer also falls within the scope of protection of the present invention.

Optionally, the silicon polymer mixture is prepared as follows: mixing the silicon polymer with water to form a 20 wt % solution, and adjusting a pH of the solution to 8-9 to obtain the silicon polymer mixture. Particularly, a percentage by mass of the silicon polymer can be adjusted according to practical application requirements.

Use of the silicon polymer or the silicon polymer mixture in preparing hair care products also falls within the scope of protection of the present application.

The present application also provides a shampoo containing the silicon polymer.

A hair mask containing the silicon polymer also falls within the scope of protection of the present application.

A hair mask, includes the following components in percentage by mass:
0.01% to 10% of the silicon polymer, 0 to 15% of a viscosity builder, 1% to 20% of a conditioner, 0 to 15% of an emollient, 0 to 10% of an emulsifier, and water to reach 100%;
wherein the viscosity builder is selected from a group consisting of cetearyl alcohol, C₁₃₋₁₆ isoparaffin and trideceth-6; wherein the conditioner selected from a group consisting of docosyl trimethylammonium chloride, octadecyl trimethylammonium chloride, hexadecyl trimethylammonium chloride, behenamidopropyl dimethylamine, stearyl trimethylammonium chloride, stearamidopropyl dimethylamine, isolauryl polyether-6, guar hydroxypropyl trimethylammonium chloride, polyquaternium-10, polyquaternium-37, decamethyl cyclopentasiloxane, and isopropyl palmitate;
wherein the emollient is selected from a group consisting of C₁₂₋₁₃ alkyl lactate, C₁₂₋₁₃ alcohol, oil-soluble lanolin, cetearyl alcohol, caprylic triglyceride, and capric triglyceride;
wherein the emulsifier is selected from a group consisting of ceteareth-25, glyceryl stearate, and PEG-100 stearate.

Preferably, the hair mask includes the following components in percentage by mass:
0.01% to 7% of the silicon polymer, 6% to 9% of the viscosity builder, 1% to 8% of the conditioner, 0 to 3% of the emollient, 1% to 3% of the emulsifier, and water to reach 100%.

Specifically, the hair mask further includes protein, a humectant, a preservative, a pH regulator and a fragrance.

For example, the above-mentioned protein may be selected from a group consisting of silk fibroin, hydrolyzed wheat protein, and hydrolyzed silk fibroin with stearic dimethylammonium hydroxypropyl group; the humectant is glycerol and/or propylene glycol; the preservative may be selected from a group consisting of phenoxyethanol, methylparaben, propylparaben, and DMDM hydantoin; and the pH regulator may be citric acid.

Compared with the prior art, the present invention possesses the following beneficial effects:

The silicon polymer of the present invention has both dimethylsilane segments and polyether segments, with pyrrolidone carboxylic acid for end capping. On one hand, the hydrophilic performance of silicon polymer can be improved, and the affinity to hair can be enhanced. On the other hand, the polyether segments cooperate with the dimethylsilane segments so that repulsive force between the hairs is provided so as to enhance the fluffiness of hair. The pyrrolidone structures at both ends of the silicon polymer have better affinity to keratin, so that the silicon polymer can be tightly attached to the damaged hair cuticle, and repair the hair cuticle, and thus the hair surface is smoother, hydrogen boding between polypeptide chains among the hair is reduced, and the softness of hair is enhanced.

### DESCRIPTION OF THE DRAWINGS

FIG. 1 is an SEM image of hair cuticle treated in Comparative Example 1.

FIG. 2 is an SEM image of hair cuticle treated in Example 1.

### DETAILED DESCRIPTION

The present invention will be further described in the following specific embodiments, but the examples do not impose any form of limitation on the present invention. Unless otherwise specified, the raw material reagents used in the examples of the present invention are conventionally available raw material reagents.

### Double Amino-Terminated Silicone Oil A1

Double amino-terminated silicone oil A1 can be prepared by the following method:
octamethylcyclotetrasiloxane (1483 g, 5 mol) and 1,3-Bis(3-aminopropyl)tetramethyldisiloxane (24.8 g, 0.1 mol) were added to a flask with protective atmosphere of nitrogen and heated to 95° C.; then 5.0 g tetramethylammonium hydroxide was added to the flask and a thermostatic reaction was performed for 10 h, then the reaction system was heated to 140° C. and vacuumized so that a low-boiling-point substance was evaporated out, the reaction system was cooled, and a colorless clear liquid was obtained and discharged, i.e. the double amino-terminated silicone oil A1.

### Double Amino-Terminated Silicone Oil A2

Double amino-terminated silicone oil A2 can be prepared by the following method:
octamethylcyclotetrasiloxane (853 g, 2.87 mol) and Bis(aminoethyl)tetramethyl disiloxane (22.8 g, 0.1 mol) were added to a flask with protective atmosphere of nitrogen and heated to 85° C.; then 1.0 g tetramethylammonium hydroxide was added to the flask and a thermostatic reaction was performed for 6 h, then the reaction system was heated to 140° C. and vacuumized so that a low-boiling-point substance was evaporated out, the reaction system was cooled, and a colorless clear liquid was obtained and discharged, i.e. the double amino-terminated silicone oil A2.

### Double Amino-Terminated Silicone Oil A3

Double amino-terminated silicone oil A3 can be prepared by the following method:
octamethylcyclotetrasiloxane (371 g, 1.25 mol) and Bis(aminodecyl)tetramethyl disiloxane (44.4 g, 0.1 mol) were added to a flask with protective atmosphere of nitrogen and heated to 85° C.; then 1.0 g tetramethylammonium hydroxide was added to the flask and a thermostatic reaction was performed for 6 h, then the reaction system was heated to 140° C. and vacuumized so that a low-boiling-point substance was evaporated out, the reaction system was cooled, and a colorless clear liquid was obtained and discharged, i.e. the double amino-terminated silicone oil A3.

### Double Amino-Terminated Silicone Oil A4

Double amino-terminated silicone oil A4 can be prepared by the following method:
octamethylcyclotetrasiloxane (1150.44 g, 3.88 mol) and Bis(aminooctyl) tetramethyl disiloxane (38.8 g, 0.1 mol) were added to a flask with protective atmosphere of nitrogen and heated to 85° C.; then 1.0 g tetramethylammonium hydroxide was added to the flask and a thermostatic reaction was performed for 6 h, then the reaction system was heated to 140° C. and vacuumized so that a low-boiling-point substance was evaporated out, the reaction system was cooled, and a colorless clear liquid was obtained and discharged, i.e. the double amino-terminated silicone oil A4.

### Silicon Polymer 1

Silicon polymer 1 can be prepared by the following method:
step S1, 150 g double amino-terminated silicone oil A1 was dissolved in 600 mL isopropanol and mixed uniformly, then a mixed solution of 22.3 g double epoxy-terminated polyether KP900 (with a molecular weight of 4400) and 80 mL isopropanol was added at 60° C. while the adding lasted for 4 h, after the adding was completed, a reaction was performed under stirring at 60° C. for 8 h, and a reaction mixture A was obtained;
step S2, 25 g anhydrous magnesium sulfate and 2.4 g dimethyl 2-methylenesuccinate were added to the reaction mixture A and mixed uniformly under stirring, after isopropanol was recovered through a reduced pressure distillation at 80° C., the reaction system was heated to 130° C. and subjected to a thermostatic reaction lasting for 8 h in a condition of vacuum and reduced pressure, and a reaction mixture B was obtained; and
step S3, the reaction mixture B of step S2 was cooled to 40° C. below, 25 g potassium hydroxide-isopropanol solution (10 wt %) was added, then the reaction system was heated to 70° C. and stirred for 3 h, after the reaction system was cooled to room temperature, 300 mL sodium chloride solution (15 wt %) was added to the reaction system, then a pH of the reaction system was adjusted to 1.5 followed by adding 500 mL ethyl acetate for extraction and separation, an organic phase was subjected to rotary evaporation in a condition of 60° C. and reduced pressure through water pump, and finally 171.2 g clear viscous liquid was obtained, i.e. the silicon polymer 1.

### Silicon Polymer 2

Silicon polymer 2 can be prepared by the following method:
step S1, 30 g double amino-terminated silicone oil A2 was dissolved in 150 mL isopropanol and mixed uniformly, then a mixed solution of 4.8 g double epoxy-terminated polyether KF400 (with a molecular weight of 2800) and 30 mL isopropanol was added at 30° C. while the adding lasted for 2 h, after the adding was completed, a reaction was performed under stirring at 30° C. for 4 h, and a reaction mixture A was obtained;
step S2, 10 g anhydrous magnesium sulfate and 0.8 g dimethyl 2-methylenesuccinate were added to the reaction mixture A and mixed uniformly under stirring, after isopropanol was recovered through a reduced pressure distillation at 80° C., the reaction system was heated to 120° C. and subjected to a thermostatic reaction lasting for 8 h in a condition of vacuum and reduced pressure, and a reaction mixture B was obtained; and
step S3, the reaction mixture B of step S2 was cooled to 40° C. below, 12 g potassium hydroxide-isopropanol solution (10 wt %) was added, then the reaction system was heated to 70° C. and stirred for 2 h, after the reaction system was cooled to room temperature, 60 mL sodium chloride solution (15 wt %) was added to the reaction system, then a pH of the reaction system was adjusted to 1.5 followed by adding 100 mL ethyl acetate for extraction and separation, an organic phase was subjected to rotary evaporation in a condition of 60° C. and reduced pressure through water pump, and finally 32.1 g clear viscous liquid was obtained, i.e. the silicon polymer 2.

### Silicon Polymer 3

Silicon polymer 3 can be prepared by the following method:
step S1, 30 g double amino-terminated silicone oil A3 was dissolved in 150 mL isopropanol and mixed uniformly, then a mixed solution of 10.1 g double epoxy-terminated polyether KF400 (with a molecular weight of 2800) and 30 mL isopropanol was added at 30° C. while the adding lasted for 2 h, after the adding was completed, a reaction was performed under stirring at 30° C. for 4 h, and a reaction mixture A was obtained;
step S2, 10 g anhydrous magnesium sulfate and 1.4 g dimethyl 2-methylenesuccinate were added to the reaction mixture A and mixed uniformly under stirring, after isopropanol was recovered through a reduced pressure distillation at 80° C., the reaction system was heated to 120° C. and subjected to a thermostatic reaction lasting for 8 h in a condition of vacuum and reduced pressure, and a reaction mixture B was obtained; and
step S3, the reaction mixture B of step S2 was cooled to 40° C. below, 20 g potassium hydroxide-isopropanol solution (10 wt %) was added, then the reaction system was heated to 70° C. and stirred for 2 h, after the reaction system was cooled to room temperature, 60 mL sodium chloride solution (15 wt %) was added to the reaction system, then a pH of the reaction system was adjusted to 1.5 followed by adding 120 mL ethyl acetate for extraction and separation, an organic phase was subjected to rotary evaporation in a condition of 60° C. and reduced pressure through water pump, and finally 38.9 g clear viscous liquid was obtained, i.e. the silicon polymer 3.

### Silicon Polymer 4

Silicon polymer 4 can be prepared by the following method:
step S1, 30 g double amino-terminated silicone oil A4 was dissolved in 150 mL isopropanol and mixed uniformly, then a mixed solution of 2.1 g double epoxy-terminated polyether KF260 (with a molecular weight of 1700) and 30 mL isopropanol was added at 30° C. while the adding lasted for 2 h, after the adding was completed, a reaction was performed under stirring at 30° C. for 4 h, and a reaction mixture A was obtained;
step S2, 10 g anhydrous magnesium sulfate and 0.5 g dimethyl 2-methylenesuccinate were added to the reaction mixture A and mixed uniformly under stirring, after isopropanol was recovered through a reduced pressure distillation at 80° C., the reaction system was heated to 120° C. and subjected to a thermostatic reaction lasting for 8 h in a condition of vacuum and reduced pressure, and a reaction mixture B was obtained; and
step S3, the reaction mixture B of step S2 was cooled to 40° C. below, 8 g potassium hydroxide-isopropanol solution (10 wt %) was added, then the reaction system was heated to 70° C. and stirred for 2 h, after the reaction system was cooled to room temperature, 60 mL sodium chloride solution (15 wt %) was added to the reaction system, then a pH of the reaction system was adjusted to 1.5 followed by adding 120 mL ethyl acetate for extraction and separation, an organic phase was subjected to rotary evaporation in a condition of 60° C. and reduced pressure through water pump, and finally 31.2 g clear viscous liquid was obtained, i.e. the silicon polymer 4.

### Examples 1-5

A hair mask, includes the following components in percentage by mass (as shown in Table 1):

TABLE 1

| | | Example | Example | Example | Example | Example |
|---|---|---|---|---|---|---|
| Component | | 1 | 2 | 3 | 4 | 5 |
| Silicon polymer 1 | | 0.7% | 0.01% | 10% | 0.05% | 7% |
| Viscosity | Cetearyl alcohol | 6% | 8% | 5% | 7% | 7.5% |
| builder | | | | | | |
| Emulsifier | Ceteareth-25 | 0 | 4% | 5% | 3% | 4.0% |
| Conditioner | Guar gum | 0 | 0.1% | 0.3% | 0.2% | 0.3% |
| | hydroxypropyl | | | | | |
| | trimethylammonium | | | | | |
| | chloride | | | | | |
| Conditioner | Polyquaternium-10 | 0.15% | 0.1% | 0 | 0.15% | 0.15% |
| Conditioner | Stearyl | 1.5% | 2% | 0.6% | 3.5% | 2.5% |
| | trimethylammonium | | | | | |
| | chloride | | | | | |
| | Behenamidopropyl | 1% | 1% | 0.4% | 3% | 2% |
| | dimethylamine | | | | | |
| Emollient | C₁₂₋₁₃ alkyl lactate | 0.4% | 0.8% | 0 | 2% | 0.8% |
| | C₁₂₋₁₃ alcohol | 0.1% | 0.2% | 0 | 0.5% | 0.2% |
| Emulsifier | Glyceryl stearate | 1% | 0.5% | 1.5% | 0 | 0.5% |
| | PEG-100 stearate | 1% | 0.5% | 1.5% | 0 | 0.5% |
| Preservative | DMDM hydantoin | 0.3% | 0.3% | 0.3% | 0.3% | 0.3% |
| ph | Citric acid | 0.3% | 0.3% | 0.1% | 0.8% | 0.7% |
| regulator | | | | | | |

| | | | | | |
|---|---|---|---|---|---|
| Fragrance | 0.5% | 0.5% | 0.5% | 0.5% | 0.5% |
| Water | 87.05% | 81.69% | 74.8% | 79% | 73.05% |

### Example 6

A hair mask, includes the same components in percentage by mass as those in Example 1, with a difference in that the silicon polymer is silicon polymer 2.

### Example 7

A hair mask, includes the same components in percentage by mass as those in Example 1, with a difference in that the silicon polymer is silicon polymer 3.

### Example 8

A hair mask, includes the same components in percentage by mass as those in Example 1, with a difference in that the silicon polymer is silicon polymer 4.

### Example 9

A hair mask, includes the following components in percentage by mass (as shown in Table 2):

TABLE 2

| | Percentage by |
|---|---|
| Component | mass |
| Silicon polymer 2 | 4.999% |
| Polyquaternium-37 | 0.76% |
| C₁₃₋₁₆ isoalkane | 0.24% |
| Trideceth-6 | 0.16% |
| Glycerol | 5% |
| Stearyl trimethylammonium chloride | 0.21% |
| Hydrolyzed silk fibroin with stearic | 0.4% |
| dimethylammonium hydroxypropyl | |
| group | |
| Propylene glycol | 0.03% |
| Methylparaben | 0.001% |
| Caprylic/capric triglyceride | 2% |
| Decamethyl cyclopentasiloxane | 2% |
| Isopropyl palmitate | 2% |
| Phenoxyethanol | 0.5% |
| Fragrance | 0.5% |
| Water | 81.2% |

### Comparative Example 1

A hair mask, includes the same components in percentage by mass as those in Example 1, with a difference in that the hair mask is free of silicon polymer 1.

### Tests

### (1) Dry-Combing Performance Test

Specific test method is as follows:
Step 1, stock solutions of the hair masks in Comparative Example 1 and Examples 1-9 were prepared into 4 wt % hair mask solutions;
Step 2, 10 bundles of damaged hair (4 g per bundle, with a length of 25 cm and a width of 2 cm) were basically washed with 10 mL of 10 wt % SLES (sodium lauryl ether sulfate);
Step 3, one bundle of the damaged hair in step 2 after washing was selected, washed with clear water (as control group), and subjected to the dry-combing performance test after natural drying, and the test were conducted 3 times; then other bundles of the damaged hair in step 2 after washing were respectively immersed into the 4 wt % hair mask solutions in step 1 and stayed still for 1 min, then the hairs were taken out and washed uniformly with tap water for 1 min, and subjected to the dry-combing performance test after natural drying, and the test were conducted 3 times respectively.

The maximum tension before/after using the product was recorded. Statistical analysis was performed using SPPS. The results were further analyzed using LSD test, and a values less than 0.05 were considered statistically significant. A difference between before and after treating with the product was analyzed, and a relative percentage difference after treating with the product was calculated. The specific test results are shown in Tables 3 and 4.

TABLE 3

| | Mean maximum | Standard |
|---|---|---|
| Group | tension/N | deviation |
| Control group | 2.93572 | 0.0772 |
| Comparative Example 1 | 1.5470 | 0.06012 |
| Example 1 | 1.0005 | 0.0872 |
| Example 2 | 0.9354 | 0.7013 |
| Example 3 | 0.5634 | 0.0658 |
| Example 4 | 0.8776 | 0.0497 |
| Example 5 | 0.4030 | 0.0500 |
| Example 6 | 0.3638 | 0.0787 |
| Example 7 | 0.7894 | 0.0711 |
| Example 8 | 0.6522 | 0.0599 |
| Example 9 | 0.3030 | 0.0500 |

TABLE 4
Percentage

| | Levene Variance | | | | |
|---|---|---|---|---|---|
| | Equality Test | | | | Relative |
| | | Variance | LSD | | Percentage |
| Group-Group | Significance | result | Significance | Result | Difference |
| Control group-Example | 642.370 | Equal | <0.001 | Significant | 65.92% |
| Control group-Example | | variance | <0.001 | Significant | 89.68% |

According to the data in Table 3, it can be seen from the comparison of Examples 1-9 and Comparative Example 1 that adding silicon polymer can reduce the maximum tension. From Examples 1-5, it can be seen that Example 5 has the smallest mean maximum tension, indicating that combination of selection and dosage of the conditioner have an impact on hair smoothness, with Example 5 having the best effect. From Examples 1 and 6-8, it can be observed that the structure of the silicon polymer has an impact on the mean maximum tension, with Example 6 having the best effect. From Examples 6 and 9, it can be seen that the silicon polymer 2 of the present invention, when combined with appropriate conditioner and used in appropriate dosage, has the smallest mean maximum tension. According to the data in Table 4, there is a significant difference between Example 1 and Example 9 compared to the control group (washed with water), indicating that the silicon polymer of the present invention can significantly improve the dry-combing performance of hair.

### (2) Morphology Measurement of Hair Cuticle

Specific test method is as follows:
Step 1, stock solutions of the hair masks in Comparative Example 1 and Example 1 were prepared into 4 wt % hair mask solutions;
Step 2, 10 bundles of damaged hair (4 g per bundle, with a length of 25 cm and a width of 2 cm) were basically washed with 10 mL of 10 wt % SLES (sodium lauryl ether sulfate);
Step 3, the damaged hair in step 2 after washing were respectively immersed into the 4 wt % hair mask solutions in step 1 and stayed still for 1 min, then the hairs were taken out and washed uniformly with tap water for 1 min, and randomly selected to perform morphology measurement via microscope after natural drying.

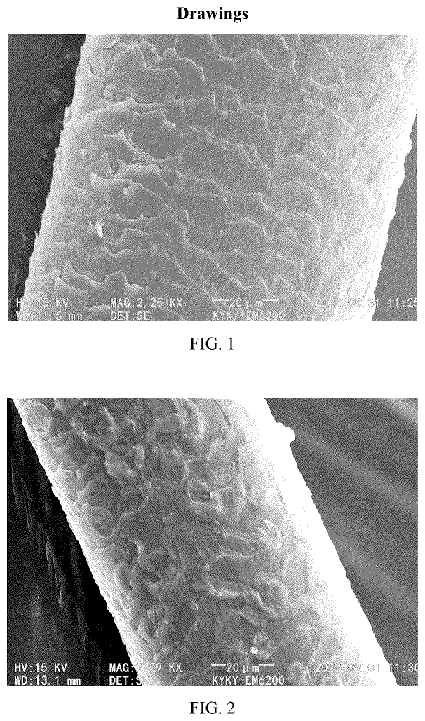

From FIG. 1, it can be seen that the hair cuticle treated with Comparative Example 1 have a larger opening degree. From FIG. 2, it can be seen that the opening degree of hair cuticle treated with Example 1 is significantly reduced, indicating a good effect in smoothing hair cuticle, further demonstrating that the silicon polymer of the present invention has a good repairing effect on hair cuticle.

The above examples of the present invention are to clearly illustrate the present invention, and not to limit the embodiments of the present invention. For those skilled in the art, different forms of changes or variations can be made based on the above description. It is not necessary and impossible to exhaustively list all implementation methods here. Any modifications, equivalent substitutions, and improvements made within the spirit and principles of the present invention shall be included within the scope of protection of the claims of the present invention.

What is claimed is:

1. A silicon polymer, having a structural formula shown as formula (I): 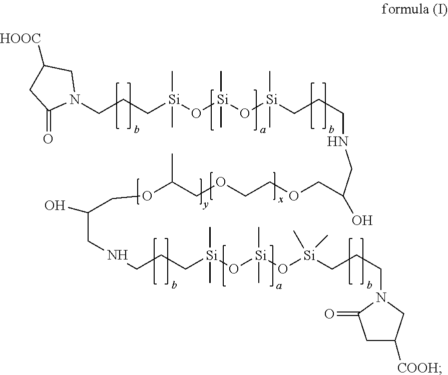
wherein 25≤a≤100, 0≤b≤8;
the formula (I) comprises a structure of 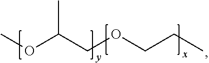
and the structure of 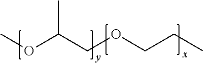
is derived from 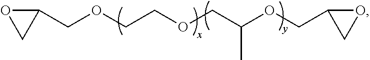
wherein the 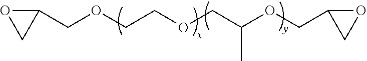
has a molecular weight of 1700-4400.

2. The silicon polymer according to claim 1, wherein x>y.

3. The silicon polymer according to claim 1, wherein x<y.

4. A preparation method for the silicon polymer according to claim 1, comprising the following steps:
step S1, dissolving a double amino-terminated silicone oil in an organic solvent, then slowly adding a double epoxy-terminated polyether at −20° C. to 60° C. to perform a thermostatic reaction for 2-8 h for obtaining a reaction mixture A; and
step S2, adding a catalyst to the reaction mixture A obtained in step S1 and mixing uniformly, then removing the organic solvent, increasing a temperature of the reaction system to 100° C. to 150° C., subjecting the reaction system to a thermostatic reaction for 2-8 h for obtaining a reaction mixture B, and subjecting the reaction mixture B to separation and purification to obtain the silicon polymer;
wherein the double amino-terminated silicone oil is prepared by a ring-opening polymerization of octamethylcyclotetrasiloxane and a reactant S, the reactant S is selected from a group consisting of Bis(aminoethyl) tetramethyl disiloxane, Bis(aminopropyl) tetramethyl disiloxane, Bis(aminobutyl) tetramethyl disiloxane, Bis(aminopentyl) tetramethyl disiloxane, Bis(aminohexyl) tetramethyl disiloxane, Bis(aminoheptyl) tetramethyl disiloxane, Bis(aminooctyl) tetramethyl disiloxane, Bis(aminononyl) tetramethyl disiloxane, and Bis(aminodecyl) tetramethyl disiloxane;
wherein the double epoxy-terminated polyether has a structural formula of 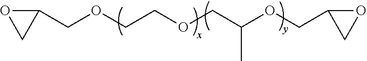
and a molecular weight of 1700-4400.

5. The preparation method for the silicon polymer according to claim 4, wherein a molar ratio of octamethylcyclotetrasiloxane to the reactant S is (12.5-50):1.

6. A silicon polymer mixture, comprising the silicon polymer according to claim 1.

7. Use of the silicon polymer according to claim 1 in preparing hair care products.

8. Use of the silicon polymer mixture according to claim 6 in preparing hair care products.

9. A shampoo, comprising the silicon polymer according to claim 2.

10. A hair mask, comprising the silicon polymer according to claim 3.
